(19) United States
(12) Patent Application Publication
(10) Pub. No.: US 2024/0123018 A1
(43) Pub. Date: Apr. 18, 2024

(54) A HERBAL BAMBOO TEA COMPOSITION AND A METHOD OF PREPARATION THEREOF

(71) Applicants: Akshya SHREE, New Delhi (IN); Dhwani SHREE, New Delhi (IN)

(72) Inventors: Akshya SHREE, New Delhi (IN); Dhwani SHREE, New Delhi (IN)

(21) Appl. No.: 18/275,118

(22) PCT Filed: Feb. 1, 2022
(86) PCT No.: PCT/IB2022/050851

§ 371 Date: Jul. 31, 2023

(30) Foreign Application Priority Data
Feb. 1, 2021 (IN) 202111004262

(51) Int. Cl. A61K 36/899; A61K 9/00

(52) U.S. Cl. CPC A61K 36/899; A61K 9/0095; A61K 2236/13; A61K 2236/15

(57) ABSTRACT

The present disclosure relates to an herbal tea composition comprising a combination of at least nine bamboo tree leaves. The said bamboo tea blend is a natural hydrant and has high ratio of elements like copper, zinc, potassium, calcium, etc. The bamboo tea composition of the present disclosure has been found to be effective in improving stress management, diabetes, cardiovascular diseases, insomnia, anemia, osteoporosis, thyroid and in maintaining skin, brain, and bone health.

### FIELD OF THE INVENTION

The present disclosure relates to an herbal tea composition and a method of preparation thereof. More particularly, the present disclosure relates to an herbal tea composition comprising dried leaves of at least nine different bamboo species. The herbal tea composition prepared is rich in other elements/micronutrients thus helping to improve immunity, stress, skin, brain, bone and body health and is useful in thyroid, osteoporosis, anemia, insomnia, diabetes and cardiovascular problems.

### BACKGROUND OF THE INVENTION

In recent years, there has been an upsurge in the clinical use of indigenous drugs. viz. herbal plants, originally used in the traditional system of medicine, are now being effectively tried in a variety of pathophysiological states. Non-specific mechanisms like restoration of normal physiological milieu and generalized increase in resistance against infections are proposed and the role of the immune system in these drug effects is suggested. Yet very few pass the test of truth and is certainly beneficial. Bamboo is one such plant.

Bamboos are well-known, evergreen, perennial flowering plants belonging to the sub-family Bambusoideae of the grass family Poaceae and found in India, China, Thailand and many parts of Asia. Bamboos are of notable economic and cultural significance, being used for building materials, as a food source, and as a versatile raw product. Bamboo, like wood, is a natural composite material with a high strength-to-weight ratio useful for structures. Most bamboo species are native to warm and moist, tropical and warm temperate climates. However, many species are found in diverse climates, ranging from hot tropical regions to cool mountainous regions and highland cloud forests.

Bamboo leaves are medium to large and are long and lanceolate in shape tapering to a sight point opposite the stem end. The deep green leaves are tough, leathery, smooth and are connected to green or brown stems that can grow up to ten centimeters a day. Typically, bamboo leaves are harvested when they are no more than thirty centimeters in length and five centimeters in width, and they have a mild, sweet taste with faint notes of green tea. There are over one thousand different Bamboo species. Bamboo leaves come from many different species and all species may have different nutrients and hence may have different medicinal properties. Bamboo leaves are extremely versatile and can be used medicinally, to preserve or flavor food. Bamboo leaves contain protein, amino acids, potassium, calcium, manganese, and iron among other nutrients. They also have a high polyphenol content and exhibit antioxidant and antibacterial effects. Bamboo leaves have a rich amalgamation of chemical compounds that make them an excellent source of curative drugs for clinical and physiological treatment in both human and livestock. Thus, bamboo leaves are frequently used in traditional medicine in India, China, Thailand, Japan and various other parts of Asia and instances of their use were recorded in pharmacopeia and other official books since the 1700s. Considered to be a cooling element to the body, bamboo leaves are believed to be able to help reduce phlegm, fever and stomach upsets. However, it is rare to find fresh bamboo leaves in stores as they are perishable and only last for a couple of days.

Bamboo tea is one of the most famous uses of bamboo leaves. Bamboo tea as known till date is a sweet, blended drink made from crushed bamboo leaves that has a myriad of medicinal uses. Bamboo tea is also rich in silica that can help reduce bloating and ease diarrhea. And for those with a bad case of flatulence, bamboo tea is a great drink. Several efforts have been made in the past that discloses that the bamboo leaves are crushed and processed to make bamboo teabags. Though, depending on the type of bamboo tree, these properties may differ.

There are abundant trials, literature, patents and commercial preparations available till date describing different types of bamboo tea either made from bamboo shoots or bamboo leaves by different processing methods. Few of them are as described below.

CN101091506 describes bamboo tea as produced from bamboo cores and leaves collected in spring and summer. The tea has good health care effect, sufficient nutrients and microelements, and good appearance.

CN1383758 covers a method for preparing health tea beverage by mixing green tea or scented tea or Oolong tea, with water and bamboo (from bamboo leaf, bamboo stem and bamboo root) of Gramineae, heating the said mixture to 100° C. for 5-10 min, boiling for 5-25 min, filtering, adding sucrose 1-6 wt. % into filtrate, mixing, sterilizing, and bottling. The said tea beverage is rich in triterpene compounds, amino acids, saccharides, etc., which are beneficial to human body. It has effects of promoting health, clearing heart fire, and relieving restlessness.

JP2008005762 describes process for preparation of bamboo tea from bamboo shoots by steaming, drying and roasting bamboo shoots.

CN104431052 discloses a bamboo leaf tea, comprising a mixture of bamboo leaf with perrin, rehmannia and green tea. The said bamboo leaf tea is described to have pure and clean taste, has the effects of promoting diuresis, cooling and clearing away summer heat.

CN110200093 discloses preparation of bamboo leaf tea from Enshi black bamboo by collecting and sun drying the leaves and then packing and storing the tea which can be brewed seven times to obtain seven different tastes and is rich in nutrients.

KR2005029422 discloses a method of making bamboo leaves tea by heating leaves of a bamboo with steam. The steamed bamboo leaves are ground to a predetermined size to obtain tea.

KR2005038220 describes a method of making bamboo leaves tea by applying the manufacturing process suitable for appearance and texture characteristic of bamboo leaves.

Thus, as mentioned in these literature and patent references, bamboo leaf tea is well-known in the art and various processes are described to prepare the tea from the leaves. However, none of the references described in the art discloses herbal bamboo tea composition obtained by preparing and processing bamboo leaves obtained by combining different bamboo species to get extraordinary beneficial effects and which can be served as a healthy drink and can also be used in the ailment of many chronic conditions and lifestyle diseases.

The primary object of the present disclosure is to provide an herbal tea composition comprising dried bamboo leaves and a method of preparing the same.

Another object of the present disclosure is to provide an herbal tea composition rich in antioxidants and other elements namely copper, zinc, potassium, calcium, silica and others.

Yet another object of the present disclosure is to provide an herbal tea composition of golden colour, having strong and pleasant fragrance, sweet textured taste and is of balanced nutritional value.

Still another object of the present disclosure is to provide an herbal tea composition useful in improving immunity, treatment/management/prevention of stress, diabetes, cardiovascular diseases, insomnia, anemia, osteoporosis, thyroid and in maintaining skin, hair, nail, brain and bone health.

### SUMMARY OF INVENTION

The present disclosure relates to an herbal tea composition comprising a combination of at least nine bamboo tree leaves. The said bamboo tea blend is a natural hydrant and has high ratio of other elements like copper, zinc, potassium, calcium, etc. The bamboo tea composition of the present disclosure has been found to be effective in improving stress management, diabetes, cardiovascular diseases, insomnia, anemia, osteoporosis, thyroid and in maintaining brain health.

In an aspect of the present disclosure, there is provided an herbal tea composition rich in antioxidants and other elements, said composition comprising of dried leaves of:
(i) Dendrocalamus longispathus 10-20% w/w;
(ii) Thyrosostachys oliveri 10-20% w/w;
(iii) Bambusa balcooa 10-20% w/w;
(iv) Bambusa tulda 10-20% w/w;
(v) Melocanna baccifera 10-20% w/w;
(vi) Bambusa balcoa 10-20% w/w;
(vii) Bambusa palhda munro 10-20% w/w;
(viii) Bambusa cacharensis var. Betu 10-20% w/w;
(ix) Bambusa cacharensis var. Pechi Betu 10-20% w/w; and
(x) Optional flavoring agent,
   wherein the composition is useful in the treatment/management/prevention of stress, diabetes, cardiovascular diseases, insomnia, anemia, osteoporosis, thyroid and in maintaining skin, brain and bone health.

In an embodiment of the present disclosure, there is provided an herbal tea composition as described herein, wherein the composition comprises dried leaves of Dendrocalamus longispathus, Thyrosostachys oliveri, Bambusa balcooa, Bambusa tulda, Melocanna baccifera, Bambusa balcoa, Bambusa pallida munro, Bambusa cacharensis var. Betu, and Bambusa cacharensis var. Pechi Betu in the ratio of 1:1:1:1:1:1:1:1:1.

In an embodiment of the present disclosure, there is provided an herbal tea composition as described herein, wherein the other elements are copper present in an amount of at least 9.75 ppm, zinc present in an amount of at least 4.02 ppm, iron present in an amount of at least 0.9 mg/100 gm, potassium present in an amount of at least 0.012, calcium present in an amount of at least 0.005 and silica present in an amount of at least 0.11%.

In an embodiment of the present disclosure, there is provided an herbal tea composition as described herein, wherein the antioxidants are polyphenols.

In an embodiment of the present disclosure, there is provided an herbal tea composition as described herein, wherein the aqueous solution or decoction of the herbal tea composition is of golden colour, having strong and pleasant fragrance, sweet textured taste and is of balanced nutritional value.

In an embodiment of the present disclosure, there is provided an herbal tea composition as described herein, wherein the herbal tea composition is added to water for 5-10 minutes for preparing a beverage.

In an embodiment of the present disclosure, there is provided an herbal tea composition as described herein, wherein the optional flavoring agent consists of dried leaves of Elettaria cardamomum, Citrus limon Brum f., Zingiber officinale, Curcuma longa, Ocimum tenuiflorum, Hibiscus rosa-sinensis and Cinnamomum tamala.

In another aspect of the present disclosure, there is provided a method for preparing the herbal tea composition as described herein, wherein the method comprises of:
(i) collecting herbal plants and washing with water;
(ii) drying of washed leaves;
(iii) crushing the dried herbal plants into powder or tiny bits or granules;
(iv) blending of dried and crushed leaves to obtain the final composition,
   wherein the composition consists of Dendrocalamus longispathus 10-20% w/w; Thyrosostachys oliveri 10-20% w/w; Bambusa balcooa 10-20% w/w; Bambusa tulda 10-20% w/w; Melocanna baccifera 10-20% w/w; Bambusa balcoa 10-20% w/w; Bambusa pallida munro 10-20% w/w; Bambusa cacharensis var. Betu 10-20% w/w; and Bambusa cacharensis var. Pechi Betu 10-20% w/w.

In an embodiment of the present disclosure, there is provided a method for preparing the herbal tea composition as described herein, wherein drying is sun drying, shade drying, freeze drying, hot-air drying, solar-assisted drying, microwave drying, microwave-vacuum drying, infrared-assisted drying, heat-pump drying, contact drying and the like.

In an embodiment of the present disclosure, there is provided a method for preparing the herbal tea composition as described herein, wherein drying causes reduction in moisture of the herbal plants up to 60%.

These and other features, aspects, and advantages of the present subject matter will be better understood with reference to the following description. This summary is provided to introduce a selection of concepts in a simplified form.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps of the process, features of the product, referred to or indicated in this specification, individually or collectively, and any and all combinations of any or more of such steps or features.

### Definitions

For convenience, before further description of the present disclosure, certain terms employed in the specification, and examples are collected here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

As such, these methods and materials fall within the scope of the appended claims. As used herein % or wt. % means weight % unless otherwise indicated. When used herein % refers to weight % as compared to the total weight percent of the phase or composition that is being discussed.

Degree Brix (° Bx) is the most common scale for measuring dissolved or soluble solids. It is used to express the concentration (% weight) or density of sugar in aqueous solutions in a wide variety of quality control operations including food and other industries.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

The present disclosure is not to be limited in scope by the specific embodiments described herein, which are intended for the purposes of exemplification only. Functionally equivalent products and methods are clearly within the scope of the disclosure, as described herein.

The present disclosure relates to an herbal tea composition comprising a combination of at least nine bamboo tree leaves. The said bamboo tea blend is a natural hydrant and has high ratio of rare nutrients like copper, zinc, potassium, calcium, etc. This tea has been found to substantially improve digestion, immunity, skin and hair conditions and general brain, bone and body health. In particular, the bamboo tea composition of the present disclosure has been found to be effective in improving stress management, diabetes, cardiovascular diseases, insomnia, anemia, osteoporosis, thyroid and in maintaining brain health.

In an aspect of the present disclosure, there is provided an herbal tea composition rich in antioxidants and other elements, said composition comprising of dried leaves of:
(i) Dendrocalamus longispathus 10-20% w/w;
(ii) Thyrosostachys oliveri 10-20% w/w;
(iii) Bambusa balcooa 10-20% w/w;
(iv) Bambusa tulda 10-20% w/w;
(v) Melocanna baccifera 10-20% w/w;
(vi) Bambusa balcoa 10-20% w/w;
(vii) Bambusa pallida munro 10-20% w/w;
(viii) Bambusa cacharensis var. Betu 10-20% w/w;
(ix) Bambusa cacharensis var. Pechi Betu 10-20% w/w; and
(x) Optional flavoring agent,
   wherein the composition is useful in the treatment/management/prevention of stress, diabetes, cardiovascular diseases, insomnia, anemia, osteoporosis, thyroid and in maintaining skin, brain and bone health.

In an embodiment of the present disclosure, there is provided an herbal tea composition as described herein, wherein the composition comprises dried leaves of Dendrocalamus longispathus, Thyrosostachys oliveri, Bambusa balcooa, Bambusa tulda, Melocanna baccifera, Bambusa balcoa, Bambusa pallida munro, Bambusa cacharensis var. Betu, and Bambusa cacharensis var. Pechi Betu in the ratio of 1:1:1:1:1:1:1:1:1.

In an embodiment of the present disclosure, there is provided an herbal tea composition as described herein, wherein the composition comprises dried leaves of Dendrocalamus longispathus, Thyrosostachys oliveri, Bambusa balcooa, Bambusa tulda, Melocanna baccifera, Bambusa balcoa, Bambusa pallida munro, Bambusa cacharensis var. Betu, and Bambusa cacharensis var. Pechi Betu in the ratio of 1:1:1:1:1:1:1:1:1, the combination being equal in weight percentage indicated at any weight percentage with 10 to percent deviation. The deviation in proportion or in weight percentage of a particular bamboo species from said 9 species would not affect the useful properties of bamboo tea as per the present disclosure.

In an embodiment of the present disclosure, there is provided an herbal tea composition as described herein, wherein the other elements are copper present in an amount of at least 9.75 ppm, zinc present in an amount of at least 4.02 ppm, iron present in an amount of at least 0.9 mg/100 gm, potassium present in an amount of at least 0.012, calcium present in an amount of at least 0.005 and silica present in an amount of at least 0.11%.

In an embodiment of the present disclosure, there is provided an herbal tea composition as described herein, wherein the antioxidants are polyphenols.

In an embodiment of the present disclosure, there is provided an herbal tea composition as described herein, wherein the aqueous solution or decoction of the herbal tea composition is of golden colour, having strong and pleasant fragrance, sweet textured taste and is of balanced nutritional value.

In an embodiment of the present disclosure, there is provided an herbal tea composition as described herein, wherein the herbal tea composition is added to water for about 5-10 minutes, for preparing a beverage.

In an embodiment of the present disclosure, there is provided an herbal tea composition as described herein, wherein the optional flavoring agent consists of dried leaves of Elettaria cardamomum, Citrus limon Brum f., Zingiber officinale, Curcuma longa, Ocimum tenuiflorum, Hibiscus rosa-sinensis and Cinnamomum tamala.

For each species of bamboo used in preparing the present herbal tea composition, two names are provided: a Latin name and a local name. In the terminology used herein, the Latin name is given first, followed by the local name which is typically given in parenthesis. For example, for the herb referred to as “Bambusa tulda (Mritinga)”, Bambusa tulda is the Latin name and Mritinga is its local name.

In some embodiments, the herbal tea composition of the present disclosure comprises a combination of bamboo species, at least one species of each of said genera.

In another aspect of the present disclosure, there is provided a method of preparing herbal tea composition. The method of the present disclosure generally comprises providing the bamboo leaves, cleaning, drying, crushing, and blending the leaves in suitable proportion to obtain bamboo tea, present herbal tea composition.

In an embodiment of the present disclosure, there is provided a method for preparing the herbal tea composition as described herein, wherein the method comprises of:
(i) collecting herbal plants and washing with water;
(ii) drying of washed leaves;
(iii) crushing the dried herbal plants into powder or tiny bits or granules;
(iv) blending of dried and crushed leaves to obtain the final composition,
   wherein the composition consists of Dendrocalamus longispathus 10-20% w/w; Thyrosostachys oliveri 10-20% w/w; Bambusa balcooa 10-20% w/w; Bambusa tulda 10-20% w/w; Melocanna baccifera 10-20% w/w; Bambusa balcoa 10-20% w/w; Bambusa pallida munro 10-20% w/w; Bambusa cacharensis var. Betu 10-20% w/w; and Bambusa cacharensis var. Pechi Betu 10-20% w/w.

In an embodiment of the present disclosure, there is provided a method for preparing the herbal tea composition as described herein, wherein drying is sun drying, shade drying, freeze drying, hot-air drying, solar-assisted drying, microwave drying, microwave-vacuum drying, infrared-assisted drying, heat-pump drying, contact drying and the like.

In an embodiment of the present disclosure, there is provided a method for preparing the herbal tea composition as described herein, wherein drying causes reduction in moisture of the herbal plants up to 60%.

In an embodiment of the present disclosure, there is provided an herbal tea composition as described herein, wherein the composition is useful as tea beverage with water, juice, alcohol or any carbonated drink to help in energy production, immunity building and imparting brain and general body health.

One aspect of the present invention is the method of preparing the leaves from each of the tree of Dendrocalamus longispathus, Thyrosostachys oliveri, Bambusa balcooa, Bambusa tulda, Melocanna baccifera, Bambusa balcoa, Bambusa pallida munro, Bambusa cacharensis (Betu) and Bambusa cacharensis (Pechi Betu); the method describing the separate and unique drying and crushing pattern as per the species to obtain the final composition.

More specifically, the preferred embodiment of the present disclosure comprises providing the blending of leaves from each species in about equal proportion to obtain final bamboo tea blend, the present herbal tea composition.

The present herbal composition of the present disclosure thus comprises of the bamboo leaves tea blend prepared from Dendrocalamus longispathus, Thyrosostachys oliveri, Bambusa balcooa, Bambusa tulda, Melocanna baccifera, Bambusa balcoa, Bambusa pallida munro, Bambusa cacharensis (Betu) and Bambusa cacharensis (Pechi Betu) species by cleaning, drying, crushing and homogenously mixing the leaves in about equal proportion to get bamboo tea, which is useful in stress management, diabetes, cardiovascular diseases, insomnia, anemia, osteoporosis, thyroid and in maintaining brain and bone health.

The biological materials of the present disclosure were collected from Simna, Tripura, India. The leaves from bamboo trees of the said species may also be used, as commercially available.

While dried material is traditionally used and preferred in herbal medicine, it must be recognized that drying of plant materials facilitates their storage, transportation and subsequent processing. Drying may not be a requirement to derive the benefits of these herbs. As such, it is understood that the present disclosure may be practiced with the corresponding quantity of the fresh plant material as well. The use of fresh plant material, sufficient to meet the requisite quantity and proportions of the extract used, come under the scope of the present claims.

The techniques taught include reducing the size of the leaves during processing. Here, the reducing process may be achieved by several ways including, but not limited to, cutting, chopping, mincing, pounding, pulverizing, macerating, milling and grinding. While one preferred way may be taught, other ways and means of achieving a reduction in size of the materials may also be used.

The leaves after collection are washed and dried separately and to a specific extent. The drying of the leaves is done under the sun or using nets followed by solar drying. The leaves are dried till the quantity of the leaves reduce to 60%. Thereafter, it is further crushed as desired and mixed homogenously. It's observed that colour of the leaves changes from green to red as they dry off. The leaves, however, retain their nutrients even after drying.

The methods commonly used for drying of leaves can be sun drying, shade drying, freeze drying, hot-air drying, solar-assisted drying, microwave drying, microwave-vacuum drying, infrared-assisted drying, heat-pump drying, contact drying and the like. The drying technique that can be used depends upon the retention of nutrients and oil in the leaves. Ideally, air drying/shade drying is considered best because it retains maximum content. The next better option is solar assisted drying or use of solar dryers that gives the same result as that of shade drying but is done faster as it uses a combined technique of sun and air drying. Pump heating and other drying techniques can also be used for commercial production in large industrial set up.

In a preferred embodiment of the invention, bamboo tea being a unique combination of leaves of at least nine bamboo species helps the body to form collagen and absorb iron and thus plays a vital role in energy production, immunity building and improvement in brain and body health.

In another preferred embodiment, the leaves from all the bamboo trees are collected, washed/cleaned, dried, crushed and further blended to form bamboo tea rich in rare nutrients.

According to one aspect of the present invention, there is provided a process for the preparation of bamboo tea comprising the steps of: (a) Collection of leaves; (b) cleaning and drying of leaves; (c) crushing of leaves and (d) blending of leaves together to obtain bamboo tea. Thus, Alternatively, the leaves from all the nine plants are collected and dried using a combination of air drying or shade drying and sun drying till the desired colour change and mass change is achieved.

Therefore, according to the present disclosure, in a method for preparation of herbal bamboo tea, dried and crushed leaves from at least nine species of bamboo tree comprising of Dendrocalamus longispathus, Thyrosostachys oliveri, Bambusa balcooa, Bambusa tulda, Melocanna baccifera, Bambusa balcoa, Bambusa pallida munro, Bambusa cacharensis (Betu) and Bambusa cacharensis (Pechi Betu) are mixed in about equal proportions. Yet, at times, about 10-20 percent of large leaves can be added to the blend to maintain the pack quantity. This addition is perfectly within the scope of the present disclosure.

The herbal Bamboo tea of the present disclosure comprising the blend of leaves of at least nine bamboo species may be prepared by adding the tea blend in water for about 5-10 minutes or making a decoction wherein it releases the extracts which can be mixed with alcohol or can be used for regular drinking either as hot beverage or cold beverage or with any other carbonated drink or can even be used for cooking.

In an embodiment of the present disclosure, the herbal Bamboo tea is consumed by leaving the tea blend in either water or alcohol or any carbonated liquid (for example, lemonade) overnight or for few hours to obtain the same beneficial effect.

Alternatively, the dried bamboo leaves from at least nine species may be appropriated in powder form (commercial leaves product of each bamboo species) and blended in about equal proportions before packing to obtain bamboo tea.

In an embodiment, the present herbal bamboo tea blend is prepared by combining leaves from at least nine bamboo species in about equal proportions by preparing the same either as hot or cold beverage with water or with carbonated drink or with alcohol for use in improving body immunity and brain, bone and body health.

In an embodiment, the present herbal bamboo tea blend is used as a nutraceutical composition, herbal tea, food supplement or cooking aid. More importantly, the present bamboo tea blend as a beverage can preferably be sipped at least two to three cups per day.

According one aspect of this invention, the bamboo tea blend prepared from combination of the leaves of at least nine bamboo species provides significant improvement in health and beauty within seven days.

According to another aspect of the present disclosure, the herbal bamboo tea blend prepared from combination of the leaves of at least nine bamboo species provides a method of relieving stress and improve sleep quality. Additionally, the present herbal bamboo tea is also employed in treating thyroid, osteoporosis, anemia, sleep disorders and cardiovascular problems. Further the herbal bamboo tea of the present disclosure helps in digestion and diabetes management. It further provides its usage in immunity building, energy production in body and improving brain health.

According, another aspect of the present disclosure, the herbal bamboo tea blend prepared from the combination of leaves of at least nine bamboo species administered as beverage comprises about 3 grams of protein and about 33% water extract and high ratio of other elements such as copper, zinc, potassium and calcium in each cup of tea and is useful as natural hydrant and helps in body repair and muscle building or it can also be made as a decoction and consumed. Further, the herbal bamboo tea beverage of the present disclosure is sipped alone or in combination with bamboo shoot pieces to obtain anti-aging and memory-enhancing effects. In addition, the herbal bamboo tea beverage of the present disclosure is sipped in combination with pineapple to gain weight management benefits. Further, herbal bamboo tea beverage of the present disclosure is co-administered along with jack fruit or parijat flowers or elephant apple to gain additional health benefits.

Although the subject matter has been described in considerable detail with reference to certain preferred embodiments thereof, other embodiments are possible.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods, the exemplary methods, devices and materials are described herein. It is to be understood that this disclosure is not limited to particular methods, and experimental conditions described, as such methods and conditions may vary.

### Example 1: Method for Preparing Herbal Tea Composition

The present disclosure provides a method for preparing herbal tea composition. The general procedure for the preparation of the herbal tea composition is described herein below:

Step 1: Collection of the leaves: The leaves are collected from the plantations at the time of the harvest or as and when the poles are cut for other usages. Or on maturing of the bamboo, the leaves fall off from the poles which can also be picked up for use.

Step 2: Cleaning and drying: Each of nine species as described herein have different texture and quality hence, leaves obtained from each species is washed and dried separately and to a specific extent. viz. leaves from Thyrosostachys oliveri (Kankaich) are thin and hence are washed and sun-dried for at least 3 days and then put into the solar dryer for at least one day. The Bambusa tulda (Mritinga) leaves are usually broad and hence are sun-dried using nets and then solar dried for at least 2 consecutive days. After drying the size or quantity of the leaves reduces by up to 60% (that is, the fresh and raw leaves weighing 10 kgs will weigh about 4 to 4.5 kgs after drying). However, the leaves from all nine species can be blended after drying to achieve maximum nutrient retention.

Step 3: Crushing: Dried leaves from each of species as described herein are inserted into a crusher (however, not all leaves go through this step; for example, Thyrosostachys oliveri (kankaich) leaves are reduced much after drying and therefore does not need crushing). During crushing any poor-quality leaves are removed to obtain the final blend in uniform ratio. The quantity of the leaves is further reduced after crushing to appear as tiny bits (or granular) or powdery in texture.

Step 4: Blending: The dried and crushed leaves are mixed in about equal proportions in each pack. That is the storage pack is sanitized and then the pack moves through all nine stations of machine where leaves from each species is added in equal proportion. Hence, for example, for a 450-gm pack, 50 gms of each species will be added simultaneously and then mixed in the end before final sealing of the pack as herbal bamboo tea composition.

The herbal tea composition of the present disclosure is prepared by boiling water in a pan, adding a teaspoon of the present herbal bamboo leaves composition and allow the solution to boil for 5-10 minutes. Cover the pan with a lid and keep the boiled aqueous mixture undisturbed for two minutes to obtain the desired results in terms of unique colour, taste and fragrance, and improved nutritional and antioxidant properties.

### Example 2: Evaluation of Nutritional and Organoleptic Properties of Herbal Tea Composition

The herbal tea composition of the present disclosure is evaluated for determining the nutritional and organoleptic properties. Silica content is determined by IS 3025 (P-35) (Indian standard 3025 part-35, method for determination of silica). Protein content was determined by IS 7219: (RA 2015) (Indian Standard 7219, Reaffirmed 2015, method for determination of protein in food). Crude fibre content present in herbal tea composition is determined by IS 10226 (P-1) (Indian Standard 10226 part-1, method for determination of crude fibre). Micronutrients namely copper, zinc and iron content in the herbal tea formulation is determined by ICP-MS (Inductively coupled plasma mass spectrometry). Potassium content in the herbal tea composition is determined by Flame Photometric analysis and calcium content is calculated by SAL/STP/Food/0309. The results obtained are shown in table 1 below. The herbal tea composition of the present disclosure has improved nutritional and organoleptic properties as compared to well-known bamboo tea composition. Bamboo Leaf Green Tea having a single specie bamboo leaf (Bambusa arundinacea) and green tea as key ingredients is used as a control.

TABLE 1
Organoleptic and nutritional properties of herbal tea composition

| Tea | Silica | Other | | | |
|---|---|---|---|---|---|
| composition | content | elements | Colour | Taste | Fragrance |
| Present herbal | >=0.11% | 1. Protein >= 6.21% | Golden | Sweet | Strong and |
| tea composition | | 2. Copper >= 9.75 ppm | | textured | pleasant |
| | | 3. Zinc >= 4.02 ppm | | taste | fragrance |
| | | 4. Iron >= 0.9 mg/100 gm | | | |
| | | 5. Crude Fiber >= 0.61% | | | |
| | | 6. Potassium >= 0.012 | | | |
| | | 7. Calcium >= 0.005 | | | |
| Bamboo Leaf | Absent | Protein <= 1 g | Green | Bitter | Strong, |
| Green Tea | | Calcium <= 1 mg | | and | raw |
| | | | | strong | fragrance |

In view of above, it is observed that in comparison to the existing herbal tea blend, the present herbal tea composition has better nutrient composition and a unique taste, colour and fragrance.

### Example 3: Evaluation of Antioxidant Activity and Total Polyphenol Content of Herbal Tea Composition

The antioxidant activity of the present herbal tea composition was determined by well-known methods (M. Oyaizu. Studies of products of browning reaction: antioxidative activities of products of browning reaction prepared from glucosamine. Japanese Journal of Nutrition, 44 (6) (1986), pp. 307-315—testing of antioxidant activity). The antioxidant activity of 200 μg/ml present herbal tea composition was recorded as 0.67±0.05. The total phenol content of the herbal tea composition was also evaluated by Quan, V.; Goldsmith, C.; Dang, T.; Nguyen, T.; Bhuyan, D. J.; Sadeqzadeh, E.; Scarlett, C.; Bowyer, M. Optimisation of Ultrasound-Assisted Extraction Conditions for Phenolic Content and Antioxidant Capacity from Euphorbia Tirucalli Using Response Surface Methodology. Antioxidants 2014, 3, 604-617.—Testing of polyphenols and antioxidant capacity. Total phenol content of the herbal tea composition was recorded as 46.2±15.6 milligrams of gallic acid equivalents per gram (mg GAE/g). The results obtained shows 5.6±1.3° Bx of total soluble solid of tea infusion, wherein tea infusion was prepared from 5 g/100 ml boiled water where infusion was carried out for 5 minutes.

### Example 4: Sensory Analysis of the Herbal Tea Composition

The herbal tea composition of the present disclosure was evaluated for various sensory parameters namely colour, aroma, aftertaste, flavour, mouthfeel and over all acceptability of present herbal tea composition. The sensory analysis was carried out by 12 trained panelists and 9-point ranking scale was used where 0 indicates worst and 9 as best. The results obtained shows that the herbal tea composition of the present disclosure is having color 6.5±0.5, aroma 4.5±0.5, aftertaste 5.5±0.3, flavour 6.0±2.1, mouthfeel 6.0±0.2 and overall acceptability 6.8±1.5.

1. An herbal tea composition rich in antioxidants and other elements, said composition comprising of dried leaves of:
(i) Dendrocalamus longispathus 10-20% w/w;
(ii)Thyrosostachys oliveri 10-20% w/w;
(iii)Bambusa balcooa 10-20% w/w;
(iv)Bambusa tulda 10-20% w/w;
(v)Melocanna baccifera 10-20% w/w;
(vi)Bambusa balcoa 10-20% w/w;
(vii)Bambusa pallida munro 10-20% w/w;
(viii)Bambusa cacharensis var. Betu 10-20% w/w;
(ix)Bambusa cacharensis var. Pechi Betu 10-20% w/w; and
(x) Optional flavoring agent,
wherein the composition is useful in the treatment/management/prevention of stress, diabetes, cardiovascular diseases, insomnia, anaemia, osteoporosis, thyroid and in maintaining skin, brain and bone health.

2. The herbal tea composition as claimed in claim 1, wherein the composition comprises dried leaves of Dendrocalamus longispathus, Thyrosostachys oliveri, Bambusa balcooa, Bambusa tulda, Melocanna baccifera, Bambusa balcoa, Bambusa polymorpha, Bambusa cacharensis var. Betu, and Bambusa cacharensis var. Pechi Betu in the ratio of 1:1:1:1:1:1:1:1:1.

3. The herbal tea composition as claimed in claim 1, wherein the other elements are copper present in an amount of at least 9.75 ppm, zinc present in an amount of at least 4.02 ppm, iron present in an amount of at least 0.9 mg/100 gm, potassium present in an amount of at least 0.012, calcium present in an amount of at least 0.005 and silica present in an amount of at least 0.11%.

4. The herbal tea composition as claimed in claim 1, wherein the antioxidants are polyphenols.

5. The herbal tea composition as claimed in claim 1, wherein the aqueous solution or decoction of the herbal tea composition is of golden colour, having strong and pleasant fragrance, sweet textured taste and is of balanced nutritional value.

6. The herbal tea composition as claimed in claim 1, wherein the herbal tea composition is added to water for 5-10 minutes for preparing a beverage.

7. The herbal tea composition as claimed in claim 1, wherein the optional flavoring agent consists of dried leaves of Elettaria cardamomum, Citrus limon Bruin f., Zingiber officinale, Curcuma longa, Ocimum tenuiflorum, Hibiscus rosa-sinensis and Cinnamomum tamala.

8. A method for preparing the herbal tea composition as claimed in claim 1, wherein the method comprises of:
(i) collecting herbal plants and washing with water;
(ii) drying of washed leaves;
(iii) crushing the dried herbal plants into powder or tiny bits or granules;
(iv) blending of dried and crushed leaves to obtain the final composition, wherein the composition consists of Dendrocalamus longispathus 10-20% w/w; Thyrosostachys oliveri 10-20% w/w; Bambusa balcooa 10-20% w/w; Bambusa tulda 10-20% w/w; Melocanna baccifera 10-20% w/w; Bambusa balcoa 10-20% w/w; Bambusa pallida munro 10-20% w/w; Bambusa cacharensis var. Betu 10-20% w/w; and Bambusa cacharensis var. Pechi Betu 10-20% w/w.

9. The method as claimed in claim 8, wherein drying is sun drying, shade drying, freeze drying, hot-air drying, solar-assisted drying, microwave drying, microwave-vacuum drying, infrared-assisted drying, heat-pump drying, contact drying and the like.

10. The method as claimed in claim 8, wherein drying causes reduction in moisture of the herbal plants up to 60%.
